(19) United States
(12) Patent Application Publication
(10) Pub. No.: US 2007/0173845 A1
(43) Pub. Date: Jul. 26, 2007

(54) Self-locking suture anchor, system and method

(76) Inventor: Andrew C. Kim, Temecula, CA (US)

Correspondence Address: NATH & ASSOCIATES, 112 South West Street, Alexandria, VA, 22314, US

(21) Appl. No.: 11/335,509

(22) Filed: Jan. 20, 2006

(51) Int. Cl. A61B 17/58

(52) U.S. Cl. 606/73

(57) ABSTRACT

A self-locking suture anchor comprises a shank with an unthreaded portion between a first, proximal thread flight and a second flight axially displaced therefrom. A suture retainer collar through which a suture can be pulled surrounds the unthreaded portion. When the self-locking suture anchor is threaded into bone, the suture is compressed between the suture retainer collar and the screw and between the bone and the screw. A drive socket is in a central cavity of the shank. A driver includes a driver head. Additional sutures may also be supported to a holding suture retainer in the central cavity. A method includes securing a suture to position sutured tissue with respect to the self-locking suture anchor or to establish a particular tension in the suture.

### FIELD OF THE INVENTION

The present subject matter relates to self-locking suture anchors comprising screws, a system for installing the suture anchors and a method of anchoring tissue.

### BACKGROUND OF THE INVENTION

Suture anchors are often used in the art to fix soft tissue in place. One common form of such a suture anchor is a surgical anchor screw having a shank with a proximal head and a distal end. The shank may be tapered. A thread comprising a plurality of thread flight can extend around the shank. Accordingly, the screw can be advanced axially by one lead into an anchoring substrate by turning the screw through one full rotation of the screw. Generally, a previously considered anchoring substrate in this regard is bone. Two basic anchoring schemes have previously been provided for anchoring tissue to bone. An anchor with sutures already loaded therein may be inserted into bone. The sutures are then secured to tissue. Alternatively, a suture or sutures that have already been secured to tissue may be secured to an anchor in a bone.

In the first type of scheme, a suture is threaded through an eyelet on the anchor, and ends of a length of suture are brought substantially into registration. In this manner, two lengths of suture, also referred to as a suture pair, can be provided secured to and extending from the anchor. Anchors having more than one eyelet or one enlarged eyelet to accommodate more than one suture pair may be provided. Commonly, the eyelet is included in a head at the proximal end of the suture anchor. The head may also commonly comprise drive means which may be rotated by a driver. In many prior art embodiments, the eyelet extends through the driving head.

U.S. Pat. No. 6,045,573 illustrates an example of a screw anchor configuration in which two suture pairs are supported to a screw anchor prior to being secured to tissue. A distal end of the screw anchor has threads which tap into a bone when the screw is rotated. A proximal end has an unthreaded head portion with radial bores serving the functions of first and second eyelets. In order to secure the screw anchor in a bone, the screw anchor is rotated to secure the threads into a bone. If only the threads are secured in the bone, the head will project above the bone. The screw anchor installation process should not be stopped at this point. If the head projects above the bone level, the projection may cause pain to the patient and tend to damage surrounding tissue. Therefore, the screw is further driven so that the threads are secured in a deeper portion of the bone, and the head is below the surface of the bone. Consequently, there are no threads securing the suture anchor to the bone for at least the depth of the head as the threads are secured more deeply in the bone.

An outer layer of bone is the cortical bone, and the bone beneath the cortical bone is cancellous bone. Cancellous bone is significantly weaker than the cortical bone. The cortical bone is relatively dense and hard. Cancellous bone may be characterized as a spongy matrix. In the above-described configuration, the screw anchor threads must be driven through the cortical bone and into the cancellous bone. When the anchor screw is driven sufficiently so that the head does not project above the bone, the unthreaded head is surrounded by the cortical bone, and the threads are secured in cancellous bone. This configuration is incapable of taking advantage of being secured in the hard, dense cortical bone.

The second type of previously known anchoring scheme anchors a suture that has already been secured to tissue. According to this second type of scheme, the anchor is generally threaded most of the way into the bone. The suture is then secured to the anchor. The anchor is then further rotated to drive the remainder of the anchor body into the bone. Initially, the suture will stay in position as the anchor is rotated. As the anchor rotates through its last turn or fraction of a turn, the suture is pulled with the rotating anchor. Accordingly, rotation of the anchor increases tautness of the suture. The unthreaded head leaves space between the head and the bone. The suture consequently moves back and forth around the head. This phenomenon is referred to in the medical literature as the windshield wiper effect. The windshield wiper effect causes cyclic loading, which weakens the suture. Accordingly, the windshield wiper effect is a shortcoming of both prior suture anchors and of surgical methods.

Surgeons have previously used sutures that extend from the anchor and project through tissue. The surgeon will then tie knots in the suture by passing a free end of the anchored suture through tissue such as a tendon before the knot is made. The knot must be made while retaining the desired tension on the suture. However, maintaining the desired suture tension increases the difficulty for the surgeon in precisely positioning an end of the tendon, making the surgery a more challenging procedure. Accordingly, a surgeon may be at a disadvantage when denied the option of using a “knotless” suture.

These prior art suture anchors have generally been rotated by elongated drivers. For precise handling of suture anchors, it is important to maintain axial alignment of the driver and the suture anchor. However, drivers and suture anchors generally form a system in which there is a limited axial extent in which a suture anchor contacts the driver. For example, U.S. Pat. No. 5,827,291 discloses a driver which contacts a suture anchor only at a driver head. Unless tight tolerances are maintained between the driver head and the driver, it is possible for the screw anchor to wobble while it is being inserted into bone.

### SUMMARY OF THE INVENTION

The present invention generally provides a self-locking suture anchor comprising a screw, a system for installing the suture anchor and a method of anchoring tissue.

In one aspect, the self-locking suture anchor comprises a shank having a proximal end and a distal end. The proximal end is formed to receive a driver and the distal end is shaped for insertion in an anchor screw substrate, preferably a bone. A thread comprising a plurality of flights is formed on the shank, with the first flight beginning at the proximal end. A second flight is displaced from the first flight by at least one lead to define an unthreaded portion of the shank. At least one suture retainer collar is positioned around the unthreaded portion. The suture retainer collar has a dimension to be captive on the shank and to permit insertion of a suture between the suture retainer collar and the shank.

In a further form, the shank comprises a central cavity in which a suture retainer is mounted for securing at least an additional suture.

In an alternative embodiment, the present invention provides a system including an anchoring screw in one of the forms described above and an elongated drive tool having a central cannula. The drive tool comprises a head to engage a drive socket and further comprises an axial alignment guide to align the anchoring screw with the drive tool.

In another embodiment, the present invention relates to a method of anchoring tissue to an anchoring substrate. According to this method, an anchoring screw is provided comprising a shank and a thread formed on the shank comprising a plurality of flights. A first portion of the thread, which may comprise one flight, begins at a proximal end of the shank. The shank has an unthreaded portion adjacent to the first portion. A suture retainer collar is positioned around the unthreaded portion. The suture retainer collar has a dimension to be captive on the shank and to permit insertion of a suture between the suture retainer collar and the shank. According to this method, a suture secured to tissue can be passed between the suture retainer collar and the shank. A free end of the suture can be held to maintain tension thereon. The screw is then introduced into the anchoring substrate and driven into the anchoring substrate up to the ring. The suture is then pulled to adjust the position of an end of the tissue secured to the suture or to adjust the tension in the suture. The remainder of the shank is then driven into the anchoring substrate to clamp the suture between the shank and the suture retainer collar and to also clamp the suture between both the shank and the first portion of the thread and the anchoring substrate.

### BRIEF DESCRIPTION OF THE DRAWINGS

The present subject matter may be further understood by reference to the following description taken in connection with the following drawings:

FIG. 1 is an elevation view, of a first embodiment of a self-locking suture anchor;

FIG. 2 is an elevation, partially in cross sectional form, of a further form of the embodiment of FIG. 1 further illustrating additional components;

FIG. 3, consisting of FIGS. 3a and 3b, illustrates a suture retainer collar in the form of a ring;

FIG. 4 is an axonometric view of a further form of suture retainer collar;

FIG. 5 consists of FIGS. 5a-5d which are respectively a lower plan view and an axonometric view of a suture collar retainer and a perspective and a plan view of a the suture collar retainer capturing a suture pair;

FIG. 6 is a cross sectional view of a self-locking suture anchor comprising a two-part screw;

FIG. 7 is an axonometric view of a driver;

FIG. 8 illustrates a self-locking suture anchor in the process of being inserted into a bone;

FIG. 9 illustrates the self-locking suture anchor of FIG. 8 engaged in a bone; and

FIGS. 10 and 11 are respectively a perspective view and a plan view of a suture anchor secured in a shoulder and holding an end of a tendon in place.

### DETAILED DESCRIPTION OF THE PREFERRED EMBODIMENTS

The preferred embodiments of the present subject matter are described with reference to the drawings presented herein. As used herein, the term “bore” refers to an axially extending recess; the term “driver” or “drive means” refers to a means which may rotate the head of the screw, anchor, or shank, and the term “lead” refers to the axial or linear distance between corresponding points on successive thread flights, particularly when the screw is rotated 360°. A hexagonal cross section, or hex, socket driver is one example of many alternative forms of a driver.

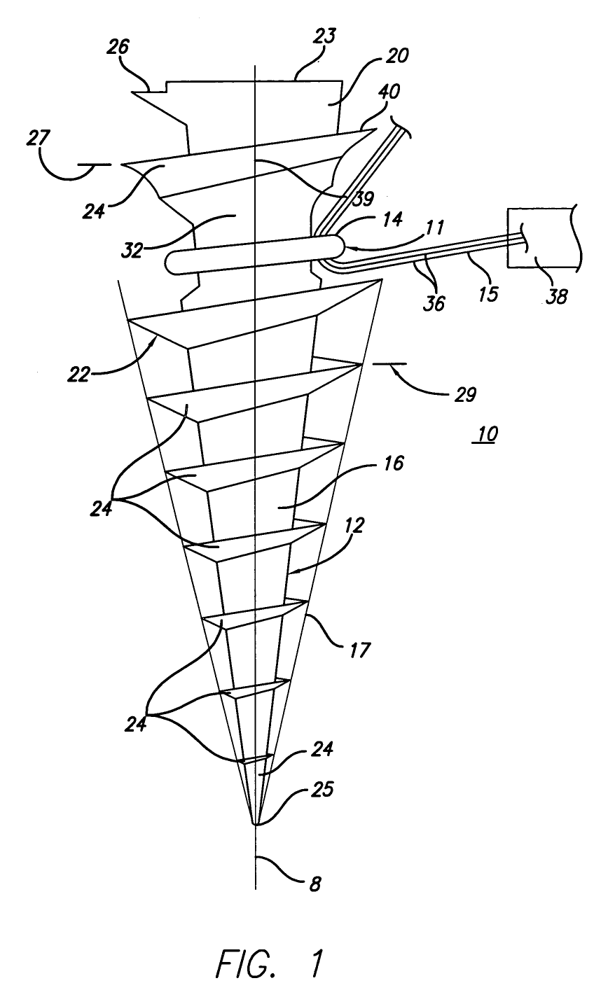

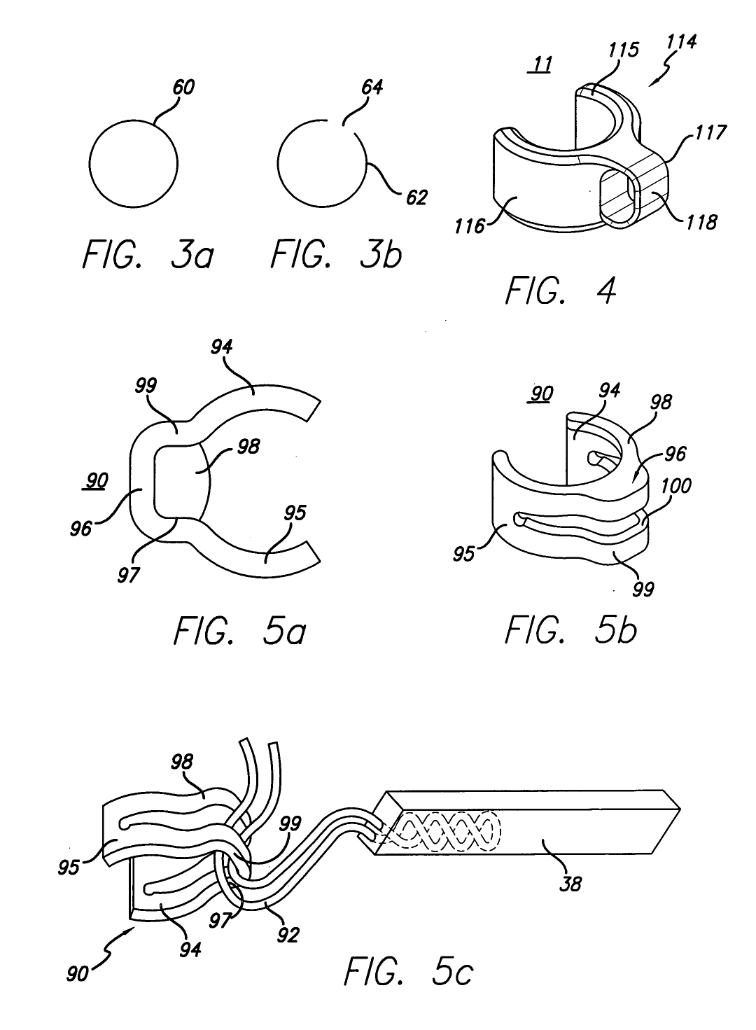

Referring to the drawings and particularly to FIG. 1, which is an elevation, a self-locking suture anchor is illustrated and designated generally by the numeral 10. The self-locking suture anchor 10 comprises a screw 12 and a suture retainer collar 11. The suture retainer collar 11 is a component constrained with respect to the screw 12 which can capture a suture 15 passed between the screw 12 and the suture retainer collar 11. When the screw 12 is fully inserted into bone, the suture retainer collar 11 will compress one portion of the suture 15 between the suture retainer collar 11 and the screw 12. The suture retainer collar 11 will compress another portion of the suture 15 between the suture retainer collar 11 and the bone. The suture retainer collar 11 in one form comprises a ring 14 captive on the screw 12. However, the suture retainer collar 11, further described below with respect to FIGS. 3 and 4, may comprise a closed curve or may comprise an open figure with a gap between ends, having overlapping ends or other disposition of the ends.

The screw 12 comprises a shank 16 having an axis of rotation 18. The shank 16 has a conical envelope 17. The shank 16 has a head 20 at a proximal end 23. The shank further has a distal end 25. In the present preferred embodiment, the shank 16 comes to a point at the distal end 25. Other types of distal ends are further contemplated herein. The screw 12 may comprise a self-tapping screw of a well-known material suitable for threading into bone. While many applications call for a self-tapping screw, in the alternative, the screw 12 could be formed as a screw requiring drilling of a pilot hole for insertion into a bone. The shank 16 may have a substantially constant taper from the proximal end 23 to the distal end 25

The screw 12 comprises a thread 22 projecting from the shank 16. The thread 22 comprises a plurality of flights 24. Each flight 24 has a circumferential extent of 360° around the shank 16 and an axial extent of one lead. In the present, preferred embodiment, one spiral thread 22 is provided. The thread 22 has a start 26 beginning substantially at the head 20 of the shank 16. The thread 22 has a first flight 27 at the proximal end 23 of the shank 16.

In accordance with a preferred embodiment of the present subject matter, a second flight 29 of the thread 22 can be displaced from the first flight 27 by providing an unthreaded portion 32 of the shank 16 intermediate the first flight 27 and the second flight 29. The axial length of the unthreaded portion 32 in a preferred, non-limiting form is one lead. More or fewer leads may be provided, and the number of leads need not be integral. The unthreaded portion 32 may form a continuous portion of the contour of the shank 16 as illustrated in FIG. 1. Alternatively, the unthreaded portion 32 may comprise a right circular cylinder or other shape. The unthreaded portion 32 has a radial extent which is less than that of the flights 27 or 29. In this manner, the suture retainer collar 11 is captured on the shank 16. In a preferred form, the unthreaded portion 32 has a sufficiently reduced radial dimension to allow the suture retainer collar 11 to fit substantially within the envelope 17 of the shank 16 and provide clearance for suture pairs 36 between the unthreaded portion 32 of the shank 16 and an inner diameter of the suture retainer collar 11.

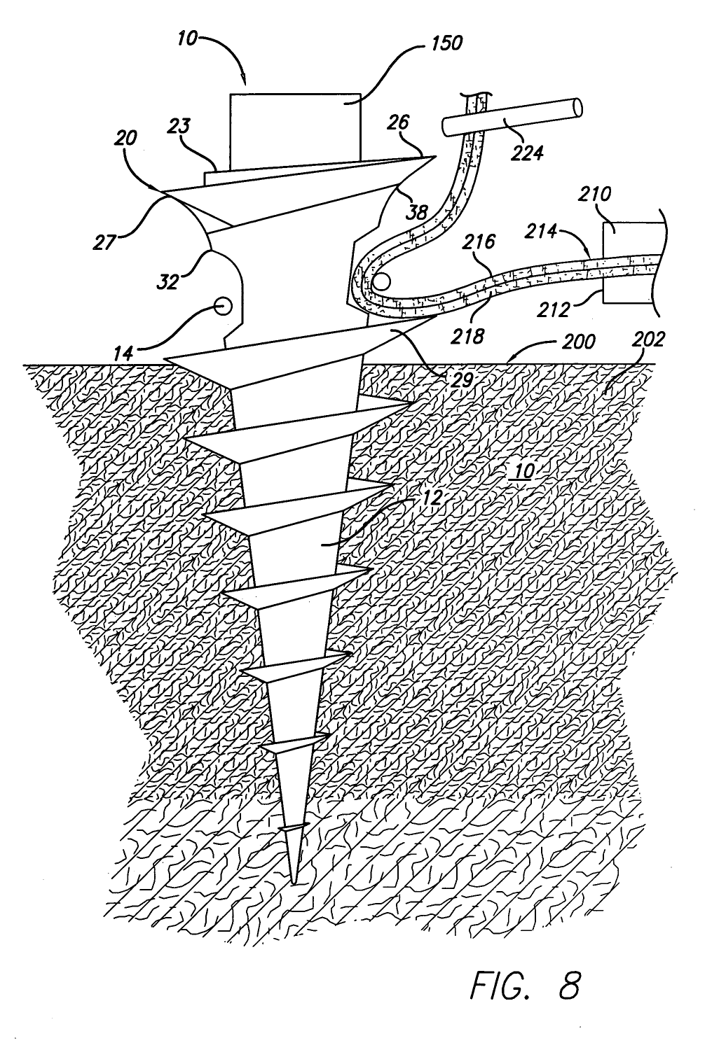

Clearance is provided between the suture retainer collar 11 and the shank 16 for insertion of a suture pair 36 therebetween. The suture pair 36 will generally comprise the suture 15 folded back on itself. The suture pair 36 may be inserted through tissue such as a tendon 38. As discussed below with respect to FIG. 8, after the screw 12 is inserted in a bone, the suture pair 36 will be held between the collar 14 and the shank 16. Additionally, the suture pair 36 will be held between the bone and the head 20. The surface of the head 20 against which the suture pair 36 is compressed is an entire surface 39 of the first flight 27. In order to prevent cutting of the suture pair 36 by a sharp edge of the first flight 27, an edge 40 of the flight 27 is blunted.

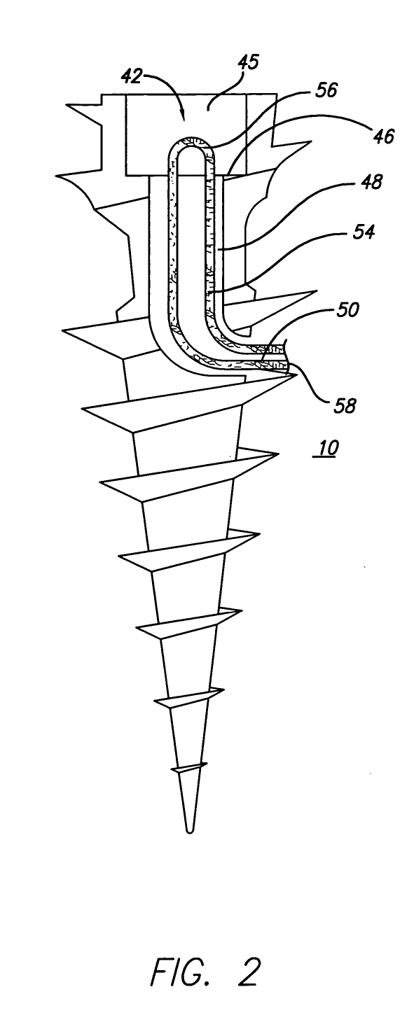

FIG. 2 is an elevation partially in cross sectional form of the self-locking suture anchor 10. As seen in cross-section, the head 20 comprises a central cavity 42. The central cavity 42 includes a drive socket 45 having a seat 46. A central channel 48 is provided extending axially toward the distal end 25 of the anchor screw 12 from the seat 46, also within the central cavity 42. A generally radial passage 50 may communicate with the central channel 48 from outside the suture anchor 10. A holding suture retainer 54 may be positioned in the central channel 48. The holding suture retainer 54 is a structure through which a suture, preferably distinct from the suture 15, can be threaded and doubled back on itself.

Many different forms of a holding suture retainer 54 may be provided. It is desirable to provide a holding suture retainer 54 which can be conveniently constructed. The holding suture retainer 54 may be formed by a length of suture material or wire formed in a loop 56. The loop 56 is held to the central channel 48 by any of various methods. In the present illustration, ends of the loop 56 outside of the screw 12 are tied in a knot 58. The knot 58 is larger than a diameter of the passage 50. When tensile force is applied to the loop 56, the knot 58 prevents the loop 56 from being pulled out of the screw 12.

Many other forms of holding suture retainer 54 may be provided. For example, a mounting socket (not shown) can be provided in a distal end of the central channel 48 that can receive legs of a “hairpin” holding suture retainer 54. Such a construction entails additional expense in materials and complexity in assembly, but serves the purpose of providing a structure to which one or more suture pairs may be retained.

FIGS. 3a and 3b illustrate forms of the suture retainer collar 11. The suture retainer collar 11 may be made of different materials to suit different applications. For example, the suture retainer collar 11 may be bioabsorbable. In the alternative, it could be made of plastic or one or more metals such as titanium or stainless steel. Another alternative is suture material. As illustrated in FIG. 3a, the suture retainer collar 11 in FIG. 1 includes a single collar member comprising the ring 14. The ring 14 is a closed loop 60. The suture retainer collar 11 may alternatively comprise a plurality of collar members, e.g. two rings 14. The cross section of the suture retainer collar 11 may have any of a wide variety of shapes, such as circular or rectangular. Edges of the suture retainer collar 11 should be rounded to avoid damaging sutures.

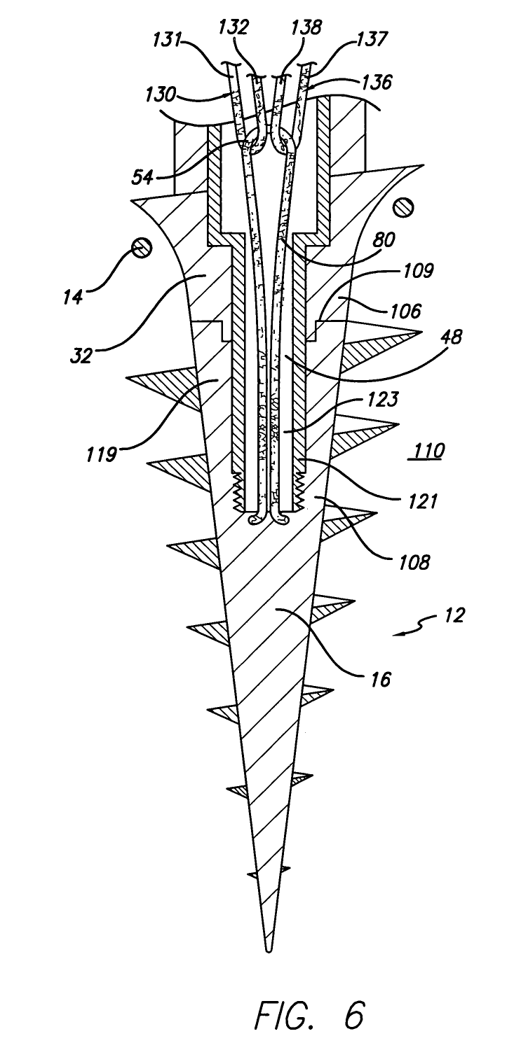

In one embodiment, the closed loop 60 may be formed by placing a length of titanium wire around the unthreaded portion 32 of the shank 16 in one or more loops and welding or tying ends of the wire to complete the suture retainer collar 11. In an alternative embodiment, the suture retainer collar 11 may be preformed. A two-piece embodiment of the shank 16, further described with respect to FIG. 6 below, allows assembly of a preformed suture retainer collar 11 around the unthreaded portion 32.

The suture retainer collar 11 may be any shape suitable for use with the shank 16. In a preferred, non-limiting embodiment, the suture retainer collar 11 is circular or oblong. An oblong shape provides greater clearance between the suture retainer collar 11 and the shank 16 for a given perimeter of the suture retainer collar 11. This facilitates the ability to insert the suture pair 36 between the suture retainer collar 11 and the shank 16. In an embodiment in which the unthreaded portion 32 is tapered, as the screw 12 is inserted into a bone, camming action of the unthreaded portion 32 against the suture retainer collar 11 can deform an oblong suture retainer collar 11 into a more nearly circular shape.

Many forms of the suture retainer collar 11 may be provided. The suture retainer collar 11 needs to provide for retaining a suture in engagement with the anchor screw 12 prior to insertion of the anchor screw 12 into a bone. The suture retainer collar 11 need not necessarily comprise a closed loop. In the embodiment of FIG. 3b, the suture retainer collar 11 comprises a loop 62 having a split 64. In to this embodiment, the suture retainer collar 11 is assembled on the unthreaded portion 32 of the shank 16 by pressing the shank 16 through the split 64. Ends of the suture retainer collar 11 may be welded together if desired.

A closed suture retainer collar 11 is preferred, as it will likely have the greatest pullout strength, i.e., the resistance to being pulled off the shank 16 when pulled on by a suture pair 36. The selection of a desired type of suture retainer collar 11 with use of a suture anchor 10 can be achieved by optimizing the pullout strength versus simplicity and cost of assembly of the suture retainer collar 11 in accordance with objectives to be accomplished.

Preferably, the suture retainer collar 11 can capture a first suture or group of sutures that is brought into engagement with the suture anchor 10. These sutures could, for example comprise sutures that have been passed though tissue. In an alternative form, additional sutures may originate from the suture anchor 10. Accordingly, suture retaining means can be provided in the central cavity 42. Sutures originating from the central cavity 42 may be threaded through tissue and then knotted or otherwise secured to position the tissue with respect to the suture anchor 10.

FIG. 4 is an axonometric view of a suture retainer 11 in the form of a suture clip 114. The suture clip 114 has legs 115 and 116 which snap on the shank 16, preferably on the unthreaded portion 32. An eye 117 within a loop 118 is secured to the suture clip 114. The suture 15 is captured by threading through the eye 117. As the screw 12 is inserted into bone, the loop 118 deforms to retain the suture 15 in place.

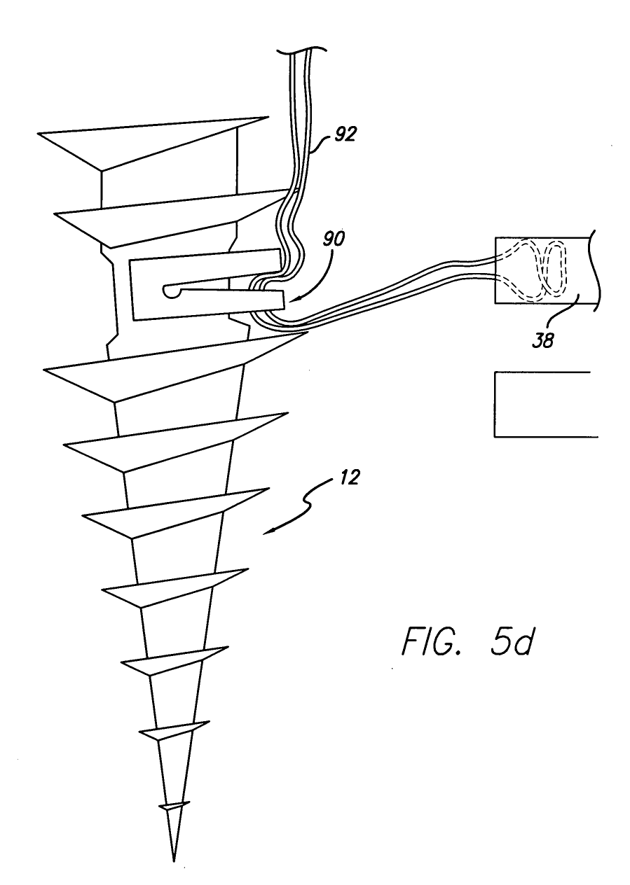

FIG. 5 consists of FIGS. 5a-5d. FIGS. 5a and 5b are respectively a lower plan view and an axonometric view of a suture retainer collar 90. The suture retainer collar 90 comprises first and second legs 94 and 95 joined by a central member 96. Upper and lower lands 98 and 99 are defined by a radial recess 100, preferably comprising a V-shaped notch through the central member 96 and through a sufficient radial depth of the legs 94 and 95 to reach the shank 16. The legs 94 and 95 are of sufficient circumference to snap onto the shaft 16, preferably on the unthreaded portion 32. The land 98 is shaped to abut the shaft 16. The land 99 is formed to provide a clearance between the shank 16 and the land 99 sufficient to allow capture of a suture or suture pair. The clearance will comprise an aperture 97 when the suture retainer collar 90 is mounted on the shank 16.

FIGS. 5c and 5d are respectively a perspective and a plan view of the suture collar retainer capturing a suture pair 92. The suture pair 92 is between a portion of said suture retainer collar 90 and the shank 16. As seen in FIGS. 5c and 5d, in one application, the suture pair 92 is secured to the tendon 38 and then threaded through the aperture 97. As the suture anchor 10 is threaded into bone, the lands 98 and 99 will be forced together to fasten the suture pair 92 in place.

FIG. 6 illustrates a suture anchor 110 comprising a proximal portion 106 and a distal portion 108 assembled together to form an anchor screw 12 with a shank 16. The proximal portion 106 and the distal portion 108 have an annular, radially extending interface 109. The suture anchor 110 may include components corresponding to those of the suture anchor 10 of FIG. 1. Accordingly, the same reference numerals are used in FIG. 6 to denote components corresponding to those in FIG. 1.

A closed suture retainer collar 11 may be used in conjunction with the suture anchor 110. Ends of the suture retainer collar 11 do not need to be tied or welded. The suture retainer collar 11 (not shown) can be placed around the unthreaded portion 32 and then captured within the screw 12 by assembling the proximal portion 106 to the distal portion 108. In one preferred embodiment, the proximal portion 106 and the distal portion 108 are press fit together. The proximal portion 106 and the distal portion 108 may be assembled other than by a press fit. In one alternative form, the proximal portion 106 includes an axial projection 119 projecting distally from the interface 109. The axial projection 119 has an inner channel defining the alignment channel 48 and an outer contour comprising a cylinder 121. The distal portion 108 has a central bore 123 to receive the cylinder 121.

FIG. 6 also illustrates a suture pair 130 having suture segments 131 and 132 and a suture pair 136 having suture segments 137 and 138 retained by the holding suture retainer 54. Each suture 130 and 136 can be inserted through the holding suture retainer 54 and then doubled back on itself to provide the ends 131 and 132 or the ends 137 and 138. Other numbers and types of sutures may also be thus retained. The sutures 130 and 136 are not initially tied or otherwise fixed to the holding suture retainer 54. The holding suture retainer 54 can keep the sutures 130 and 136 in place while tension is maintained on both ends of the suture 130 or 136. The suture 130 or 136 may, if desired, be removed by pulling on one end thereof. If the suture segment 131 of suture 130, for example, is threaded through tissue and the suture segments 131 and 132 are then anchored or knotted, the suture 130 can be retained by the holding suture retainer 54.

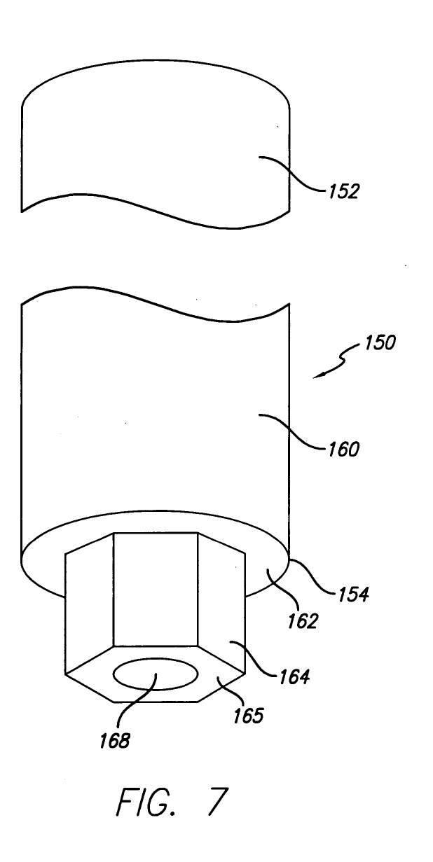

FIG. 7 is an axonometric view of a driver 150 for use with the self-locking suture anchor 10 or 110. The driver 150 and the suture anchor 10 or 110 comprise a system in which components cooperate to precisely and reliably anchor a suture. The driver 150 has a proximal end 152 and a distal end 154 and comprises an elongated drive shaft 160. The drive shaft 160 in one form comprises a right circular cylinder. Alternatively the drive shaft could be ribbed on its outer circumference in the manner of a screwdriver handle or may comprise other surfaces used for other forms of driving tools. The distal end 154 of the driver 150 can have a radially extending surface 162 which will abut the proximal end 23 of the suture anchor 10 or 110. A driver head 164 can project axially from the driver 150 and has a contour and depth to fit the drive socket 45. The driver head 164 can have a distal, radially extending surface 165. The seat 46 (FIG. 2) of the drive socket 45 may act as an axial stop means for the radially extending surface 165. The driver 150 can further have a central, axial cannula 168. A suture 130, for example, can be threaded through the holding suture retainer 54, and the ends 131 and 132 drawn through the cannula 168 and secured at the proximal end 152 of the driver 150. In this manner, any suture extending through the cannula 168 is kept in place while the suture anchor 10 or 110 is being inserted into a bone. Also, an axial force is applied to keep the driver 150 inserted in the central cavity 42.

In accordance with a further embodiment of the present subject matter, an alignment rod (not shown) could be provided projecting from the driver head 164. According to this embodiment, the alignment rod would be received in the central channel 48. This provides an extended axial engagement of the driver 150 in the central cavity 42, minimizing any wobbling of the suture anchor 10 or 110 with respect to the driver 150. However, use of an alignment is generally unnecessary since the driver head 164 and the drive socket 45 (FIG. 2) can be made with sufficiently close tolerances for reliable interaction.

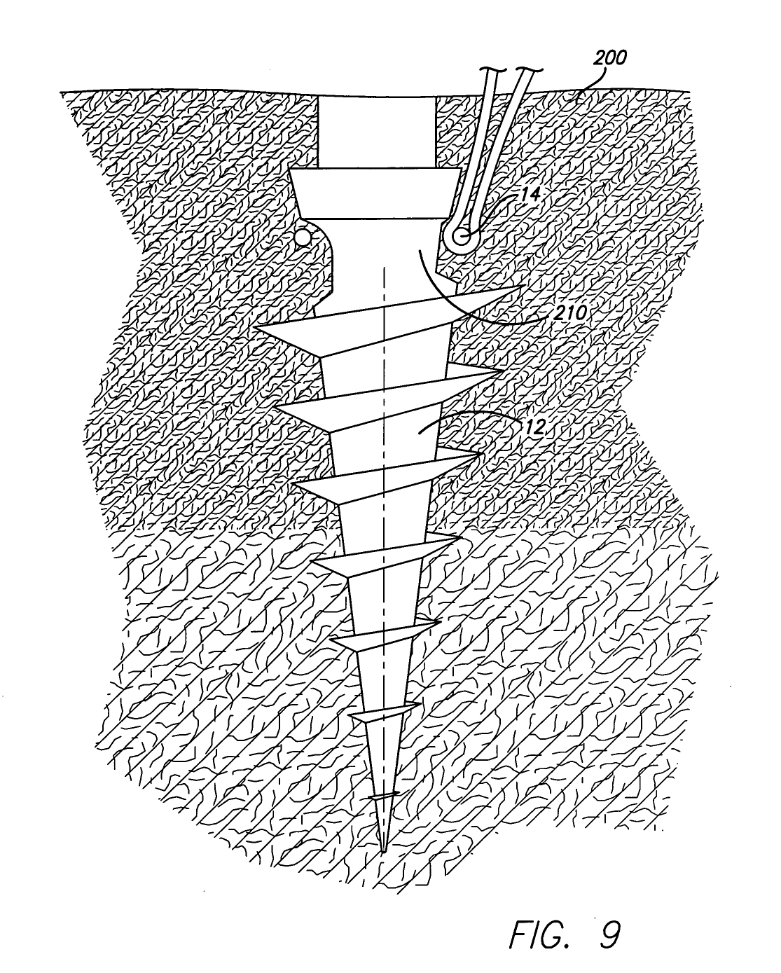

FIGS. 8 and 9 are elevations, partially in cross sectional form, illustrating a use of the suture anchor 10. The suture anchor 110 (FIG. 6) may be used in the same manner. In this embodiment, the suture anchor 10 can be inserted in a bone 200. In a preferred embodiment, the suture anchor 10 or 110 may be received completely or substantially completely in the cortical bone 202. The suture that is to be anchored may be, for example, a suture 214 that has been inserted through a tissue 210 having an end 212. This embodiment enables a surgeon to select a position of the end 212 or a tension to be imposed on the suture 214. Types of tissue 210 typically operated on in the current context are tendons and ligaments.

In the illustration of FIG. 8, the suture 214 is threaded through the tissue 210. The suture 214 has ends 216 and 218 which extend from the tissue 210. In order to anchor the suture 214, the ends 216 and 218 are drawn between the suture retainer collar 11 and the screw 12, and more particularly the unthreaded portion 32. Preferably the ends 216 and 218 are drawn across a lower, i.e. distal, side of the suture retainer collar 11 and then drawn through the collar 14. Tension is then maintained on the suture 214. This may be done, by non-limiting example, by grasping the ends 216 and 218 with a clamp 224. The clamp 224 may be held by a person, for example.

With the suture 214 thus positioned, the surgeon uses the driver 150 (FIG. 7) to rotate the screw 12. As the screw 12 rotates, the proximal end 23 advances axially toward the bone 200. The suture 214 remains stationary. As the screw 12 advances into the bone 200, the diameter of the unthreaded portion 32 bearing against the suture retainer collar 11 increases, while the clearance between the suture retainer collar 11 and the unthreaded portion 32 decreases. Once the screw 12 is in the bone 200 up to the second flight 29 and before the bone 200 compresses the suture retainer collar 11 against the surface 38, the surgeon pulls on the suture 214 to a desired tension. In many situations, the surgeon will bring the tissue 210 all the way to the screw 12 and bone 200. The surgeon then finishes driving the screw 12 into the bone 200 to result in the arrangement shown in cross section in FIG. 9. The suture 214 is buried in the bone 200 along with the suture retainer collar 11 and the proximal end 23 of the screw 12. This locks the suture 214 to the bone 200, providing a knotless anchor.

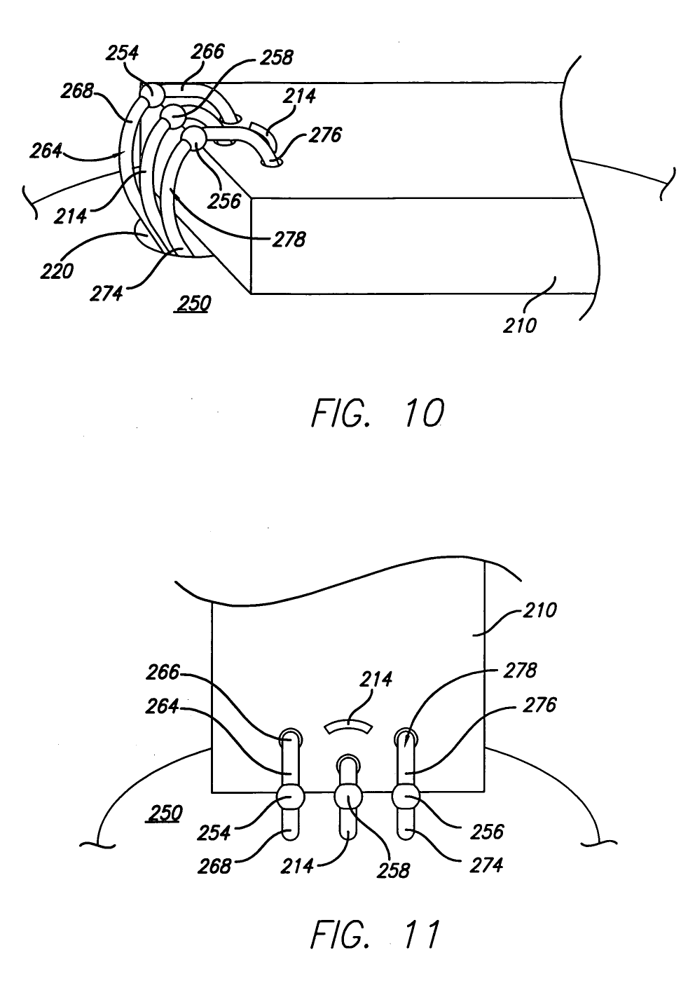

After the suture 214 is anchored, free ends will be present. The surgeon may make another pass through the tissue 210 if desired. With or without making an extra pass, the surgeon may knot the free ends of the suture 214, as further described below with respect to FIG. 10, to form a security knot. This security knot may be made without concern about maintaining the suture 214 in tension because the tissue 210 is already anchored via the suture 214. This form of anchoring the suture 214 is useful, for example, in arthroscopic repair of the rotator cuff of the shoulder. The procedure illustrated with respect to FIG. 8 allows for passing a free suture through the soft tissue 210 and then anchoring the free suture. This is an easier task for a surgeon than passing a suture which is secured to an anchor already in the bone through the tissue. In that case, when making the knot, the surgeon must be concerned about maintaining proper tensioning on the suture and placement of the tissue while making the knot.

FIGS. 10 and 11 are respectively a perspective view and a plan view of a suture anchor 220 secured in a shoulder 250 in the manner described with respect to FIGS. 9 and 10. A suture 214 is anchored knotlessly. A free end of the suture 214 comprises a remainder 214′. The remainder 214′ is used to make an additional know fixation 258. Further illustrated are additional knots 254 and 256 formed after the suture 214 is knotlessly anchored. The knots 254 and 256 are made with first and second anchored tension sutures 264 and 274 which pass through the loop 56 of the holding suture retainer 54 (FIG. 2). The suture 264 has ends 266 and 268. The suture 274 has ends 276 and 278. The sutures 264 and 274 are secured by the loop 56 in the central cavity of the suture anchor 220 in the manner illustrated with respect to FIG. 10. One of the ends 266 or 268 of the suture 264 is passed through the tissue 210, and the ends 266 and 268 are knotted, with excess length beyond the knot being cut off. Similarly, one of the ends 276 or 278 of the suture 274 is passed through the tissue 210, and the ends 276 and 278 are knotted, with excess length beyond the knot being cut off. The additional knots may be formed on the suture remainder 214′.

The present subject matter being thus described, it will be apparent that the same may be modified or varied in many ways. Such modifications and variations are not to be regarded as a departure from the spirit and scope of the present subject matter, and all such modifications are intended to be included within the scope of the following claims.

What is claimed is:

1. A self-locking suture anchor, comprising:
a) a shank having a proximal end and a distal end, said proximal end formed to receive a drive means and said distal end shaped for insertion in a bone;
b) a thread formed on said shank, said thread comprising a plurality of flights, wherein a first thread portion flight begins at said proximal end of said shank and a second thread portion is displaced on said shank from said first thread portion;
c) an unthreaded portion on said shank intermediate said first thread portion and said second thread portion; and
d) a suture retainer collar constrained with respect to said shank and positioned to keep the sutures stationed between said unthreaded portion and said suture retainer collar while the screw shank is rotated and driven to compress a suture passed between said suture retainer collar and said shank or against said suture retainer collar when said shank is inserted in bone.

2. A self-locking suture anchor according to claim 1, wherein said first thread portion comprises a single flight.

3. A self-locking suture anchor according to claim 1, wherein said unthreaded portion is untapered.

4. A self-locking suture anchor according to claim 1, wherein said first flight comprises a thread with dulled edges.

5. A self-locking suture anchor according to claim 1, wherein said suture retainer collar comprises a ring and wherein said unthreaded portion and said ring are dimensioned such that said ring fits within an envelope of said shank.

6. A self-locking suture anchor according to claim 1, wherein said suture retainer collar is made of metal.

7. A self-locking suture anchor according to claim 1, wherein said suture retainer collar is made of plastic.

8. A self-locking suture anchor according to claim 1, wherein said suture retainer collar is made of suture material.

9. A self-locking suture anchor according to claim 1, wherein said shank comprises first and second axially aligned components assembled to retain said suture retainer collar.

10. A self-locking suture anchor according to claim 1, wherein said shank is unitary.

11. A self-locking suture anchor according to claim 1, wherein said suture retainer collar comprises a collar positioned around said shank.

12. A self-locking suture anchor according to claim 11, wherein said collar comprises a closed ring.

13. A self-locking suture anchor according to claim 11, wherein said collar comprises a ring having an opening.

14. A self-locking suture anchor according to claim 11, wherein said collar comprises first and second legs and wherein said legs are deformable to permit mounting of said ring on said shank.

15. A self-locking suture anchor according to claim 14 wherein said collar further comprises an eye located adjacent said legs to receive a suture.

16. A self-locking suture anchor according to claim 1, wherein said suture retainer collar comprises at least a first and a second collar.

17. A self-locking suture anchor according to claim 1, wherein said shank comprises a root tapered from said proximal end to said distal end.

18. A self-locking suture anchor according to claim 11, wherein said suture retainer collar forms a loop that is elongated in one dimension, wherein said loop is formed into a substantially circular form by camming action of said root as said shank is advanced into a bone.

19. A self-locking suture anchor according to claim 17, wherein said shank comprises a drive socket communicating with said proximal end.

20. A self-locking suture anchor according to claim 19 further comprising a central cavity in said shank.

21. A self-locking suture anchor according to claim 20, further comprising a holding suture retainer mounted in said central cavity to hold tension sutures.

22. A self-locking suture anchor according to claim 21, wherein said cavity comprises a central bore and a passage communicating with an exterior of said shank.

23. A self-locking suture anchor according to claim 22, wherein said holding suture retainer comprises a loop having ends extending through said passage and secured in a stop knot at the exterior of said shank.

24. A self-locking suture anchor according to claim 1, further comprising a central cavity in said shank.

25. A self-locking suture anchor according to claim 20, further comprising a holding suture retainer housed in said central cavity.

26. A self-locking suture anchor system comprising:
a) a shank having a proximal end and a distal end, said proximal end formed to receive a drive tool and said distal end shaped for insertion in a bone;
b) a thread formed on said shank, said thread comprising a plurality of flights, wherein a first flight begins at said proximal end of said shank;
c) a second flight displaced on said shank from said first flight to define an unthreaded portion of said shank;
d) a suture retainer collar positioned around said unthreaded portion of said shank and having a dimension and configuration to be captive on said shank and to permit insertion of a suture between a portion of said suture retainer collar and said shank;
e) a central cavity in said shank communicating with said proximal end and comprising a drive socket;
f) a holding suture retainer mounted in said central cavity; and
g) an elongated drive tool having a head to engage said drive socket and further comprising a central cannula.

27. A self-locking suture anchor system according to claim 26, wherein said drive tool is engaged in said shank and further comprising a suture secured to said holding suture retainer and extending through said central cannula, said suture securing said shank to said drive tool.

28. A self-locking suture anchor system according to claim 27, wherein said suture is threaded through said holding suture retainer and doubled back on itself.

29. A method of anchoring tissue to an anchoring substrate comprising:
a) providing an anchoring screw comprising a shank, a thread formed on said shank, said thread comprising a plurality of flights, wherein a first flight begins at said proximal end of said shank, said shank having an unthreaded portion adjacent to said first flight, and a suture retainer collar positioned around said unthreaded portion of said shank and having a dimension and configuration to be captive on said screw and to permit insertion of a suture on or between said suture retainer collar and said shank;
b) passing a suture secured to tissue between a portion of said suture retainer collar and said shank;
c) holding a free end of said suture to maintain tension thereon;
d) introducing the anchoring screw into an anchoring substrate;
e) driving the anchoring screw into the anchoring substrate until the anchoring screw is inserted up to the suture retainer collar;
f) pulling the suture to adjust a position of an end of the tissue secured to the suture or tension in said suture;
g) driving the screw completely into the anchoring substrate to clamp the suture between the shank and the suture retainer collar and also clamp the suture between the shank and first thread and the anchoring substrate; and
h) releasing the free end of the suture.

30. A method according to claim 29, wherein the anchoring substrate comprises bone.

31. A method according to claim 28, wherein the tissue comprises tendon.

32. A method according to claim 30, further comprising providing a suture retainer collar comprising first and second rings positioned around said unthreaded portion of said shank.

33. A method according to claim 32, wherein passing the suture between each ring and the shank comprises passing two suture strands through each ring.

34. A method according to claim 33, further comprising providing a holding suture retainer in the central cavity of the shank and providing at least an additional suture secured to said holding suture retainer.

35. A method according to claim 34, further comprising securing the at least an additional suture to the tissue.
